(19) United States
(12) Patent Application Publication
(10) Pub. No.: US 2009/0038610 A1
(43) Pub. Date: Feb. 12, 2009

(54) INHALATION DEVICE FOR PROVIDING A MIST OF NEBULISED LIQUID MEDICAL SOLUTION TO A USER

(75) Inventors: Bo Bogh, Roskilde (DK); Erling Rasmussen, Billund (DK); Jorn Eskildsen, Torring (DK)

Correspondence Address: CANTOR COLBURN, LLP, 20 Church Street, 22nd Floor, Hartford, CT, 06103, US

(73) Assignee: EQUINE NEBULIZER APS, Hørsholm (DK)

(21) Appl. No.: 12/090,598

(22) PCT Filed: Oct. 18, 2005
(86) PCT No.: PCT/DK2005/000670

§ 371 Date: Sep. 2, 2008

(51) Int. Cl. A61M 11/00

(52) U.S. Cl. 128/200.16

(57) ABSTRACT

A device is disclosed including ultrasonic nebulising means for providing a mist of a liquid medical solution for the treatment of diseases in the lungs, such as asthma or the like, to a human or animal user, such as a horse, the device including a mist reservoir enclosure for receiving the mist produced by the nebulising means, whereby a mist may be produced to fill the enclosure prior to inhalation thereof. The mist is therefore kept ready for inhalation for a longer period of time instead of being produced only in connection with the actual inhalation, and a more efficient and precise inhalation with finer droplets and less waste of the medical solution is obtained.

### TECHNCIAL FIELD

The present invention related to a device for providing a mist of a liquid medical solution for the treatment of e.g. conditions and diseases in the respiratory system, in particularly in the lungs, such as asthma or the like, to a human or animal user, such as a horse.

### BACKGROUND

Ultrasonic nebulising devices for producing a mist of a liquid medical solution is well known in the art and is disclosed e.g. in U.S. Pat. No. 3,561,444, in U.S. Pat. No. 4,319,155, and in U.S. Pat. No. 5,666,946. These devices are either operated where the user activates the ultrasonic nebuliser and inhales the produced mist, or the nebuliser is activated e.g. by detection of an inhalation and the mist is forced into the respiratory system of the user by means of a ventilator as disclosed in U.S. Pat. No. 6,530,370.

It is not without problems to employ the known devices with users that cannot or will not cooperate in the administration of the liquid medical solution and inhale at the correct time, such as infants, humans suffering from mental conditions or diseases, and animals. This leads to waste of medicine, which is nebulised but not inhaled, and it is therefore difficult or impossible to determine the amount of medicine administered to the user.

### BRIEF SUMMARY OF THE INVENTION

The invention provides a device for providing a mist of a liquid medical solution to a user that cannot or will not cooperate in inhaling the mist, wherein the waste of the liquid medicine is reduced or eliminated.

The invention further provides a device where the distribution of droplet sizes in the mist is more advantageous, i.e. that the number of larger droplets inhaled is reduced.

The invention provides a device comprising ultrasonic nebulising means for producing a mist of the liquid medical solution and a mist reservoir enclosure for receiving the mist produced by the nebulising means, whereby a mist may be produced to fill the enclosure prior to inhalation thereof. The mist is therefore kept ready for inhalation for a longer period of time instead of being produced only in connection with the actual inhalation, and the production and the inhalation of the mist is thus made independent. The whole portion of medical solution may thus be inhaled without waste, which also has the positive implication that the environment near the device is less polluted with wasted medical solution.

Thus, the present invention relates to a drug nebulising device for providing a mist of a liquid medical solution to a user, the device comprising ultrasonic nebulising means for producing a mist of the liquid medical solution, which are well known per se, a mist reservoir enclosure for receiving the mist produced by the nebulising means, the mist reservoir having first valve means for selectively closing a fluid flow connection between the enclosure and an inhalation outlet to a user of the device and second valve means for selectively closing a fluid flow connection between an inlet and the enclosure, the first and second valves being closed prior to inhalation, and control means for controlling the operation of the ultrasonic nebulising means so as to establish a mist concentration within the enclosure prior to inhalation by the user through the inhalation outlet.

The ultrasonic nebulising means typically comprises a cup for containing the liquid medical solution, a transducer in contact with the cup and exiting means for exiting the transducer to an ultrasonic rate so that a mist is formed from the liquid medical solution contained in the cup.

The valves are preferably flow activated, so that they normally are closed and open when a negative pressure is applied to the inhalation outlet, i.e. when the user inhales.

By providing the mist to a reservoir enclosure prior to the inhalation thereof, it is furthermore obtained that larger droplets drop to the bottom of the enclosure or get stuck on the internal walls of the enclosure, so that the size distribution of the droplets in the inhaled mist becomes more advantageous, i.e. that the contents of larger droplets is reduced and the average size of the droplets is reduced, which is preferable as the smaller or finer droplets may enter deeper into the lungs of the user where the medical solution should be applied.

In a preferred embodiment, the device further comprises detection means for detecting an inhalation of the user and providing an output accordingly to the control means, wherein the control means is adapted to control the operation of the ultrasonic nebulising means in response to said output. The detection means may be a flow detector for detecting an airflow out through the inhalation outlet or an airflow in through the inlet, typically from the environment. Alternatively, the detection means detects a lower pressure in the enclosure due to the inhalation through the inhalation outlet. In a further alternative embodiment, the inhalation is detected externally to the device, such as on the user, e.g. a movement of the diaphragm or an activation of a ventilator connected to the user. In a further alternative, the detection means detects the position of one the valves allowing a flow of air into or from the enclosure.

The ultrasonic nebulising means may in one embodiment be operated continuously by the control means to produce mist when said output indicates an inhalation by the user. This may be advantageous for users with a low respiration force, such as children or infants, whereby a mist is present in a high concentration in the air inhaled from the device during all of the inhalation.

Alternatively, the operation of the ultrasonic nebulising means is halted by the control means not to produce mist when said output indicates an inhalation by the user. This embodiment is advantageous for users with a high respiration volume and/or applying a strong suction force, such as a horse, where the flow through the inhalation outlet may cause mist with larger droplets to escape from the ultrasonic nebulising means if it was operated during the inhalation.

The control means may in a further preferred embodiment be arranged to operate the ultrasonic nebulising means alternating between active periods where mist is produced and inactive periods so as to establish and maintain a substantially constant mist concentration within the enclosure prior to inhalation by the user through the inhalation outlet. The lengths of the active periods and of the inactive periods are in one embodiment predetermined, e.g. a 15 to 20 second starting active period, followed by alternating periods of 5-8 second inactive periods and 2-5 second active periods. This is advantageous over a continuous production of mist, which may cause a heating of the liquid medical solution leading to possible deterioration of the solution. Also, the concentration of the mist should not be exaggerated, as the fusion of droplets will increase at higher concentrations, leading to the creation of a higher number of larger droplets. Furthermore, power from the electrical batteries or the like used to energise the nebulising means is saved. It is presumed that the complete dose of the medical solution is inhaled by the user over 10 to 20 inhalations through the inhalation outlet. In a further preferred embodiment of the invention, the control means of the device are arranged to adjust the length of the alternating active and inactive periods in accordance with the pattern of inhalations through the inhalation outlet, so that the active periods are extended in case of a higher frequency inhalation pattern and are shortened in case of a lower frequency inhalation pattern. The length of the inhalation periods may also be included by the control means for adjustment of the length of the alternating active and inactive periods.

Also, after four minutes without a flow detected, the nebulisation is terminated by the control means according to a particular embodiment.

In an alternative embodiment, the device comprises a sensor for detecting a measure of the mist concentration in the enclosure and producing an output to the control means accordingly, wherein the lengths of the active periods and of the inactive periods are determined by the control means from said output. The sensor may e.g. detect a measure of the opaqueness of the air inside the enclosure. Another possibility is to provide a sensor measuring the dielectric properties of the air inside the enclosure, i.e. a measure of the capacitance of the air with the mist. The capacitance is strongly dependent on the content of mist, which normally mostly consist of water.

The nebulising means is preferably arranged in a lower part of the enclosure. In particular, the nebulising means may comprise a cup for containing the liquid medical solution, the cup being arranged so that droplets formed on the inner walls of the enclosure will gather in the cup due to gravity. The enclosure is preferably of a tapering shape towards the cup.

The inhalation outlet is advantageously arranged in an upper part of the enclosure where the fewest larger droplets will be present. This is particularly advantageous if the nebulising means at the same time is arranged in a lower part of the enclosure, so that the distance between the two is large, such as between 3 and 20 centimetres. For use by human infants, a distance in the range of 3 to 8 centimetres may be advantageous, for human adults the distance may be within the range of 6 to 12 centimetres, whereas for larger animals, such as horses, a distance of 10 to 20 centimetres is preferred.

The device may further comprise air circulation means, such as a fan, arranged for circulation of the air with mist inside the enclosure. Hereby, the finer droplets are transported from the nebulising means where the droplets are generated and into the enclosure, whereas the larger droplets are less susceptible to an airflow and will remain near the nebulising means and probably return to the reservoir for the liquid medical solution.

The air circulation means may in particular be arranged to circulate air with mist from an upper part of the enclosure and to the lower part of the enclosure whereby mainly finer particles are present in the circulating air, thus preventing that larger droplets are circulated which could take them to the inhalation outlet, as could be the case in an intake for circulation air was arranged in the lower part of the enclosure.

The volume of the enclosure is preferably within the range of 50 to 5000 cm³. For human infants a volume in the range of 50 to 500 cm³, in particular within the range of 100 to 350 cm³ have shown to be advantageous. For adult humans a volume in the range of 400 to 1200 cm³, in particular within the range of 600 to 900 cm³ have shown to be advantageous. For larger animals, such as horses, a volume in the range of 1000 to 5000 cm³, in particular within the range of 1500 to 3500 cm³ have shown to be advantageous.

The mist reservoir enclosure is in a particularly preferred embodiment at least partly made in a transparent or translucent material so as to allow for visual inspection of the contents from the exterior of the device. The device may further comprise at least one light source means, such as one or more coloured light diodes arranged for illuminating the interior of the mist reservoir enclosure while the device is in operation. Thereby, it is achieved that a reliable visual detection of the correct operation of the device is obtained, i.e. that a mist is present in the reservoir and that a flow of the air with the mist takes place. In case of malfunction, e.g. that the nebulising does not operate, the cup for the medical solution is empty or the mist does not flow out through the inhalation outlet during an inhalation, the status is easily detected visually by the user or the person assisting the user. Furthermore, the light may function as an indicator of the activation of the on-switch of the device.

The present invention also relates to the use of a device as disclosed above for administering a dose of a liquid medical solution to a user and to the method of providing a nebulised liquid medical solution to a user.

### BRIEF DESCRIPTION OF THE DRAWINGS

An embodiment of the present invention is shown in the enclosed drawing, of which

FIG. 1 is a vertical cross section along a symmetry plane of a device according to the invention, and

FIG. 2 is a view in perspective of the cross sectioned device of FIG. 1.

### DETAILED DESCRIPTION OF INVENTION

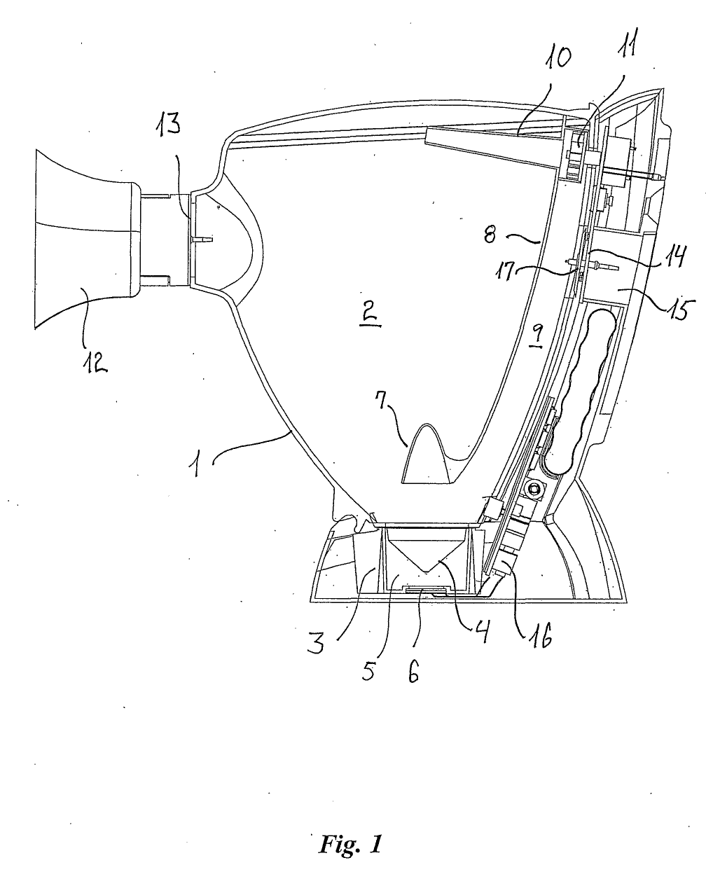

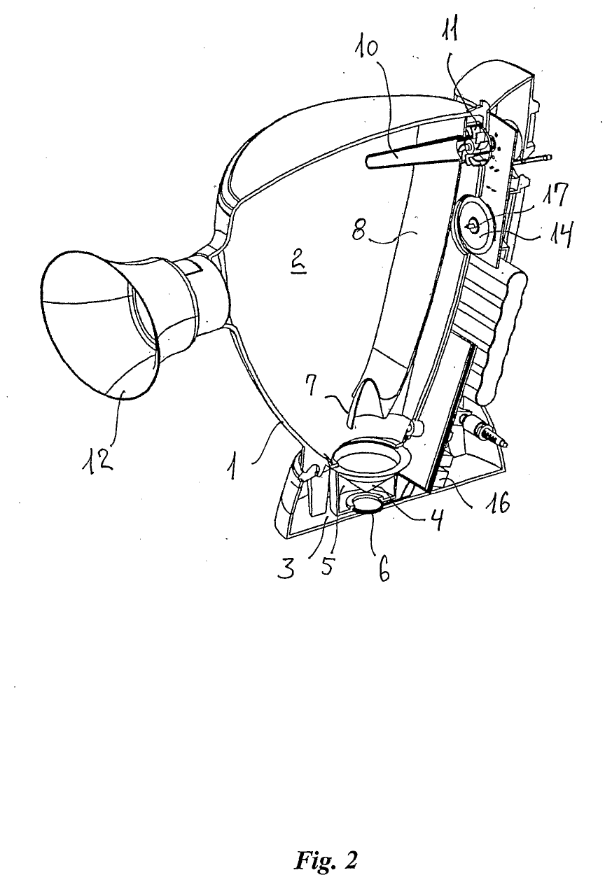

The device shown in FIGS. 1 and 2 comprises a container 1 made in a transparent plastic material and defining a mist reservoir enclosure 2. In the bottom of the enclosure 2, an ultrasonic nebuliser 3 is arranged having a cup 4 for containing the liquid medical solution, a transducer 5 in the form of water in contact with the cup 4 and exiting means 6, a piezo-electric 2.4 MHz exciter for exiting the transducer 5 to an ultrasonic rate, so that a mist is formed from the liquid medical solution contained in the cup 4. Above the cup 4, a dome 7 is arranged on an arm 8, the dome 7 will during operation of the device catch large droplets generated mainly near the centre of the liquid surface of the solution in the cup 4 and return the large droplets to the cup 4 for repeated nebulisation. The arm 8 has also the function of being a separating wall that defines a flow channel 9 at the back of the enclosure 2, the flow channel 9 being used for guiding a flow of air with mist in the enclosure from the top thereof via a frustro-conical tube 10. The flow of air is driven by a radial fan 11 arranged in the flow channel 9 and the flow is guided to be parallel to the liquid surface in the cup 4, whereby in particular the finer droplets are moved by the flow of air and are brought upwards in the enclosure 2.

The user places the inhalation mouthpiece 12 (or in case of a device designed for horses: the nasal inhalation mask) in or around the mouth and the lower pressure caused by the inhalation opens the inhalation outlet valve 13, which otherwise is closed. The lower pressure in the enclosure 2 opens the inlet valve 14, which is otherwise closed and is arranged to allow air from the environment to enter the enclosure 2. The inlet opening 15 opens to the flow channel 9 so that the incoming air is guided along the flow path and past the cup 4 before it is admixed with the air with mist inside the enclosure. Thus, a premature lowering of the mist concentration in the air to be inhaled is prevented.

The enclosure 2 is formed so that the distance between the cup 4 and the inhalation outlet 12 is as large as possible and the cup 4 is situated at the bottom of the enclosure 2 whereas the outlet 12 is situated at the top thereof to ensure that the most advantageous size distribution of the mist droplets is present in the inhalation air, i.e. a small amount of larger droplets and a large amount of fine droplets. The large droplets released from the liquid surface in the cup 4 are either captured by the dome 7, drops down in the enclosure 2 due to gravitational effect on the droplets or collides with the surface of the inner wall of the enclosure 2 due to the flow of air created by the radial fan 11, where the finer droplets tend to avoid contact with the wall surface. In all cases, the larger droplets will return to the cup 4 for repeated nebulisation.

For operation of the device, a portion of a liquid medical solution is placed in the cup 4 and the controller 16 operates the exciter 6 for a first period of time of 10 seconds in order to fill the enclosure 2 with a mist. There after, the exciter is alternately paused for 5 seconds and operated for 5 seconds in order to maintain a substantially constant concentration of the mist inside the enclosure 2 until an inhalation through the mouthpiece 12 takes place. The inhalation outlet valve 13 as well as the inlet valve 14 open due to the suction from the user, and a detector 17 on the inlet valve 14 detects the change of position of the valve 14 and provides an output to the controller 16 accordingly. The controller 16 halts the operation of the exciter 6 during the inhalation so as to prevent that larger droplets are generated and transported away from the cup 4 and are inhaled. When the inhalation stops, the establishment and maintenance of the mist concentration in the enclosure 2 is repeated. The operation of the device is halted when the cup 4 is empty, i.e. when all of the liquid medical solution has been nebulised. This may be detected by the controller 16 from the electrical parameters of the exciter 6.

The present device is made for use with horses, but similar devices may be designed for use with infants, mentally or physically disabled humans, etc.

1. A drug nebulising device for providing a mist of a liquid medical solution to
a user, the device comprising:
ultrasonic nebulising means for producing a mist of the liquid medical solution, a mist reservoir enclosure for receiving the mist produced by the nebulising means, the mist reservoir having first valve means for selectively closing a fluid flow connection between the enclosure and an inhalation outlet to a user of the device and second valve means for selectively closing a fluid flow connection between an inlet and the enclosure, the first and second valves having means for opening the valves when the user inhales and otherwise keep the valves closed, and
control means for controlling the operation of the ultrasonic nebulising means so as to establish a mist concentration within the enclosure prior to inhalation by the user through the inhalation outlet.

2. A device according to claim 1, further comprising detection means for detecting an inhalation of the user and providing an output according to the control means, wherein the control means is adapted to control the operation of the ultrasonic nebulising means in response to said output.

3. A device according to claim 2, wherein the ultrasonic nebulising means is operated continuously by the control means to produce mist when said output indicates an inhalation by the user.

4. A device according to claim 3, wherein the operation of the ultrasonic nebulising means is halted by the control means not to produce mist when said output indicates an inhalation by the user.

5. A device according to claim 1, wherein the control means are arranged to operate the ultrasonic nebulising means alternating between active periods where mist is produced and inactive periods so as to establish and maintain a substantially constant mist concentration within the enclosure prior to inhalation by the user through the inhalation outlet.

6. A device according to claim 5, wherein the lengths of the active periods and of the inactive periods are predetermined.

7. A device according to claim 5, wherein the lengths of the active periods and of the inactive periods are determined by the control means from a measured pattern of inhalation frequency and/or inhalation period lengths.

8. A device according to claim 5, further comprising a sensor for detecting a measure of the mist concentration in the enclosure and producing an output to the control means accordingly, wherein the lengths of the active periods and of the inactive periods are determined by the control means from said output.

9. A device according to claim 1, wherein the nebulising means is arranged in a lower part of the enclosure.

10. A device according to claim 9, wherein the nebulising means comprises a cup for containing the liquid medical solution, the cup being arranged so that droplets formed on the inner walls of the enclosure will gather in the cup due to gravity.

11. A device according to claim 1, wherein the inhalation outlet is arranged in an upper part of the enclosure.

12. A device according to claim 1, further comprising air circulation means arranged for circulation of the air with mist inside the enclosure.

13. A device according to claim 12, wherein the air circulation means are arranged to circulate air with mist from an upper part of the enclosure and to the lower part of the enclosure.

14. A device according to claim 1, wherein a volume of the enclosure is within the range of 50 to 5000 cm³.

15. A device according to claim 1, wherein the mist reservoir enclosure is at least partly made in a transparent or translucent material so as to allow for visual inspection of the contents from the exterior of the device, and wherein the device further comprises at least one light source means arranged for illuminating the interior of the mist reservoir enclosure while the device is in operation.

16. A device according to claim 1 configured for administering a dose of a liquid medical solution to a user.
